(19) United States
(12) Patent Application Publication
(10) Pub. No.: US 2005/0163856 A1
(43) Pub. Date: Jul. 28, 2005

(54) Abuse-resistant sustained-release opioid formulation

(75) Inventors: Ann Maloney, Dublin, OH (US); Debra Marie Murwin, Orient, OH (US); Michael Jay Schobelock, Grove City, OH (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Roxane Laboratories, Inc., Columbus, OH (US)

(21) Appl. No.: 11/087,154

(22) Filed: Mar. 23, 2005

Related U.S. Application Data
(63) Continuation of application No. 10/264,020, filed on Oct. 3, 2002.
Application No. 10/264,020 is a continuation-in-part of application No. 10/085,597, filed on Feb. 27, 2002, now abandoned.
Application No. 10/264,020 is a continuation of application No. 09/626,584, filed on Jul. 27, 2000, now abandoned.
(60) Provisional application No. 60/146,298, filed on Jul. 29, 1999.

(51) Int. Cl. A61K 31/485; A61K 9/14

(52) U.S. Cl. 424/486; 514/282

(57) ABSTRACT

A method for reducing the abuse potential of an oral dosage form of an opioid extractable by commonly available household solvents said method comprising combining a therapeutically effective amount of the opioid compound, or a salt thereof, a matrix-forming polymer and an ionic exchange resin.

### RELATED APPLICATIONS

This application is a continuation-in-part application of patent application Ser. No. 10/085,597 filed Feb. 27, 2002, which was a continuation of patent application Ser. No. 09/626,584, filed Jul. 27, 2000, which claims, as the present application, priority to Provisional Patent Application Ser. No. 60/146,298, filed Jul. 29, 1999, the disclosures of all of which are incorporated herein in their entirety.

### BACKGROUND OF THE INVENTION

1. Field of Invention

The present invention relates to a controlled-release opioid delivery composition that is resistant to extraction of the opioid with commonly-available solvents. More particularly, the present invention is directed to a controlled-release opioid formulation, capable of providing sustained, prolonged, repeat and/or delayed release, which provides resistance to extraction of the opioid using commonly-available solvents. Such formulations are useful for decreasing the potential for abuse. The formulation employs an ion exchange resin in conjunction with a hydrophilic matrix and the opioid.

2. Background of the Related Art

Opioids comprise a diverse group of drugs, natural and synthetic, that have, in varying degrees, opium- or morphine-like properties and bind to one of several subspecies of opioid receptors. These drugs produce their major effects on the central nervous system and bowel. Effects of the opioids are remarkably diverse, including analgesia, drowsiness, changes in mood and alterations of the endocrine and autonomic nervous systems. Opioid analgesics comprise the major class of drugs used in the management of moderate to severe pain. nervous systems. Opioid analgesics comprise the major class of drugs used in the management of moderate to severe pain.

One of the effects of opioid administration is the ability of such drugs in some individuals to alter mood and feeling in a manner so as to provide a desirable sense of “well-being” dissociated from therapeutic ameliorative effects. This mood-altering effect is found by some individuals to be extremely pleasurable, so much so, that some users after repeated administration develop a craving for re-administration of the opioid. The intensity of this craving may range from a mild desire to use the drug, to a preoccupation with its procurement and use, not for its therapeutic ameliorative effects, but rather for its mood-altering effects. In the latter case, the opioid becomes the central fixation in a state commonly referred to as “drug abuse,” a term used to describe the usage of any drug in a manner which deviates from approved medical or social patterns within a given culture. When the drug abuse involves overwhelming involvement with the use of the drug, securing its supply, and a high tendency to relapse into drug use after its withdrawal, an “addiction” is said to have developed. Often an addict will administer opioids in the face of self-harm.

A consequence of the repeated use of many opioids is the development of “tolerance” and, in some cases “physical dependence.” “Tolerance” refers to a phenomenon when after repeated administration of the drug, a given dose of the drug produces a decreased effect, or conversely, when increasingly larger doses of the drug must be administered to obtain effects observed with the original dose. “Physical dependence” references an altered physiological state produced by the repeated administration of the drug that necessitates the continued administration of the drug to prevent the appearance of a stereotypical syndrome, the withdrawal or abstinence syndrome. A person may also develop “psychological dependence” which is characterized by a drug-seeking behavior directed towards achieving euphoria and escape from daily life.

Tolerance does not develop uniformly to all of the actions of opioid drugs. Typically, however, tolerance will develop to the euphorigenic and other CNS depressant effects. Tolerance to a number of opioid drugs can develop with remarkable rapidity. However, the rate at which tolerance develops depends on the pattern of use. It is known that it is possible to obtain desired analgesic and sedative effects of most opioids from doses in the therapeutic range for nearly an indefinite period of time. However, when there is more or less continuous drug action, tolerance may develop. Thus in the addict who primarily seeks to get a “rush” or maintain a state of dreamy indifference (a “high”), the dose of the opioid to reach such a state must be constantly increased. In general, there appears to be a high degree of cross-tolerance between drugs with morphine-like actions, although cross-tolerance may not be seen when the opioids act through different opioid receptors. Tolerance to opioids largely disappears when a user undergoes “withdrawal” from the drug.

The time required to produce physical dependence on any opioid depends on a number of factors, including dosage schedule, route of administration, and the physiological profile of the opioid. The degree to which function in the CNS is altered by the drug, and the continuity of this alteration, appear to be very important in the development of physical dependence.

The development of clinically observable physical dependence gives rise to the possibility of reinforcement of drug abusive behavior based on administration of the drug operating to alleviate “withdrawal distress.” However, whether withdrawal symptoms are clinically observable depends on several factors including the criteria used for withdrawal symptoms, the sensitivity of the technique used to detect withdrawal, and the rate at which the drug is removed from its site of action. Withdrawal symptoms from opioid agonist administration may be aggravated when opioid antagonists are administered. For example, long-acting opioids, such as methadone, produces withdrawal symptoms that are slow in onset and generally less severe than short-acting opioids. However, when an antagonist is given to a person displaying dependence on a long-acting opioid, a severe withdrawal syndrome ensues.

It is known that a protracted opioid abstinence syndrome may develop subsequent to withdrawal of certain opioids and the condition can last for weeks. Such syndrome is characterized by physiological and psychological abnormalities that give the person a subjective sense of “not being quite right.” Such syndrome may be alleviated by administration of an opioid, predisposing one to relapse by creating a period of increased vulnerability during which the effects of opioids are especially reinforcing.

Common symptoms of opioid withdrawal include abdominal cramps, anorexia, chills alternating with excessive sweating, goose flesh, hyperexcitability, hyperirritability, increased heart rate, lachrymation, nausea, pupillary dilation, muscle spasms, and rhinorrhea. Withdrawal symptoms may manifest gradually or precipitously, and typically begin to occur 24-48 hours after the last dose of the opioid.

The abuse potential of any particular opioid relates to a number of factors including the capacity of the drug to induce euphoria, patterns of side-effects when the drug is used at supra-therapeutic doses, the distress caused by withdrawal of the drug after dependence has developed, the ability of the drug to suppress withdrawal symptoms caused by withdrawal from other opioids, and physical characteristics of the drug, such as solubility.

Three basic patterns of opioid abuse have been identified in the United States. One involves individuals whose drug use begins in the context of medical treatment and initially obtain their drug through medical channels. Another involves persons who begin their drug use with experimental or “recreational” drug use and progress to more intensive drug use. Lastly, there are users who begin using drugs obtained from medical channels or through recreational drug channels, but later switch to oral opioids obtained from organized addiction treatment programs.

A number of schemes have been introduced to reduce the incidence of drug abuse with drugs capable of altering mood and producing states of euphoria. Primary among these schemes in the United States is a legal infrastructure that controls the manufacture and distribution of such drugs. In the United States, the vast majority of opioid drugs having clinically useful and approved effects are restricted to dispensing on a prescription-only basis. Most of these drugs are “scheduled” as “controlled drugs”, such that distribution of the drug is subject to strict controls and overview. The idea behind scheduling opioid drugs as “controlled” is to ensure that the drugs are dispensed only for the amelioration of legitimate therapeutic maladies, and not for any mood-altering effect “high” or euphoria that may be produced by the drug when used in supra-therapeutic doses or administered by non-approved routes of administration.

While the scheduling of opioids as “controlled drugs” has greatly reduced abuse of the drugs, it has not been entirely successful. For example, some persons who are legitimately prescribed the drugs sometimes divert the drugs to persons seeking their procurement for “recreational uses.” These “recreational drug users” are frequently found to be willing to pay significant sums of money for the drugs. In other cases, certain health professionals, unfortunately, have been found to be culprits in the non-approved distribution of opioid drugs. When health-care professionals are involved, there is often little belief on behalf of the health professional that the patient seeking the drug wishes to use the drug for a therapeutic reason. Of course, there are also “rogue laboratories” that prepare opioid drugs without Food and Drug Administration (“FDA”) oversight and distribute such drugs to abusers.

It is believed, however, that the most widely used diversion technique at the street level is doctor shopping. Individuals, who may or may not have a legitimate ailment requiring a doctor's prescription for controlled substances, visit numerous doctors, sometimes in several states, to acquire large amounts of controlled substances they abuse or sell to others.

Scheduling of opioid drugs has also had the unintentional side-effect of causing physicians, fearful of being accused of permitting drug abuse, to prescribe sub-optimal doses of opioids to patients in need of them, and to prescribe less effective drugs to patients that are not similarly scheduled. This is particularly true with respect to the treatment of cancer patients who are frequently given sub-optimal pain control because of fears with respect to the “addictive nature” and “legal controls” surrounding approved opioid drugs. There is a growing recognition in the medical community that a large number of patients suffer from the undertreatment of pain. Among the reasons frequently cited as causative of undertreatment are: (1) the failure to prescribe enough drug at the right dosage interval to reach a steady-state threshold commensurate with the pain relief needed; (2) failure of patients to comply with a given dosage regimen; and (3) the reluctance of many physicians to prescribe analgesics categorized as controlled drugs based on often unfounded concerns of future addiction and fear of regulatory review of the physician's prescribing habits. For example, it has been reported that with respect to cancer pain, a large percentage of cancer patients suffer debilitating pain despite treatment with analgesics (Cleeland et al., N. Eng. J. Med. 330 (1994) 592-596).

Little can be done to stop the illegitimate production of opioid drugs and their distribution. However, a number of approaches or procedures, apart from the legal controls described, have been developed to dissuade the misuse of opioids drugs by patients. These approaches have been developed by legitimate pharmaceutical companies for FDA-approved uses.

Most attempts to curtail abuse of opioids by pharmacological methods have centered upon the inclusion of an “opioid antagonist” along with the opioid agonist. “Opioid antagonists” are opioids that appear to bind to receptors bound by opioid agonists but initiate little agonistic action. They typically block or reverse all of the effect of opioid agonists. These opioid antagonists may include naloxene, naloxone, nalorphine, naltrexone and nalmefene.

For example, a drug known as Valoron®N (Goedecke), that comprises tilidine (50 mg) and naloxene (4 mg), has been available in Germany for the management of severe pain. Likewise, U.S. Pat. No. 4,457,933 to Gordon et al. teaches the reduction in the oral abuse potential of the analgesics oxycodone, propoxyphene and pentazocine by combining the analgesic with naloxone in a specific range. Naloxone is combined with the selected analgesic a ratio of 2.5-5:1 part. U.S. Pat. No. 6,228,863 to Palermo et al. teaches the reduction of the abuse potential of oral dosage forms of opioid analgesics by selecting the particular opioid agonist and antagonist pair, and the concentrations of the same such that the antagonist cannot be easily extracted from the agonist (at least a two-step extraction process being needed to separate the drugs—see also, WO 99/32120). The antagonist is in such a concentration that the combination will cause an aversive effect in a physically dependent human subject but not in a naive individual (See also, WO 99/32119).

Abuse of opioids by the oral route is significant. However, another significant problem for opioid abuse appears to be the abuse of the drugs by parenteral administration, particularly by injection. Rapid injection of opioid agonists is known to produce a warm flushing of the skin and sensations in the lower abdomen described by addicts as similar in intensity and quality to sexual orgasm. The state, known alternatively as a “rush,” “kick,” or “thrill,” typically lasts for only about 45 seconds but is found extremely pleasurable to addicts. It is known in the art that individuals will extract solid dosage forms of opioids and then inject the same to attain such a state.

Presently available pharmacological methods for dissuading the extraction of oral opioids to obtain opioids typically also center upon the incorporation of opioid antagonists, or mixed opioid agonist-antagonists, with the therapeutic opioid agonist. In most systems the dose of opioid antagonist is not orally active but will block the effects desired by abusers of the agonist drug, or mixed agonist-antagonist drug, when the drug is dissolved to obtain the agonist (or mixed agonist-antagonist drug) and the opioid is subsequently administered parenterally.

For example, a commercially available drug Talwin®NX (Sanofi-Winthrop) contains pentazocine (a benzomorphan derivative that has opioid agonist actions and weak opioid antagonistic activity) in conjunction with the naloxone (basically a pure opioid antagonist). Talwin®NX is indicated for the relief of moderate to severe pain. The amount of naloxone in the preparation is low enough that it has no action when taken orally and does not interfere with the desired agonist activities of pentazocine. However the concentration of naloxone in the preparation is high enough that when extracted from the preparation along with the pentazocine and injected into an individual that its has profound antagonistic action to the pentazocine agonist activities. Similarly, a fixed combination of buprenorphine (a semisynthetic, highly lipophilic opioid derived from thebaine, having 25 to 50 times the potency of morphine) with naloxone is available in New Zealand as Temgesic®NX for the treatment of pain.

U.S. Pat. No. 3,773,955 to Pachter et al. describes orally effective analgesic compositions which contain from about 0.1 mg to about 10 mg naloxone with the opioid analgesic. Upon extraction of the composition, parenteral administration is dissuaded, as the dose of naloxone is high enough to prevent the production of analgesia, euphoria or physical dependence from the opioid analgesic. WO 01/58447 describes a controlled-release composition which contains an opioid agonist and opioid antagonist that provides an analgesic amount of the opioid agonist over 8 hours along with an amount of opioid antagonist to attenuate a side effect of the opioid agonist. WO 01/58451 discloses an oral dosage form comprising an opioid agonist in releasable form and a sequestered opioid antagonist which is substantially not released when the dosage form is administered intact but is released upon tampering. As indicated above WO 99/32120 further describes selecting the opioid agonist and antagonist with respect to physical properties so as to require at least a two-step extraction process to separate the opioid agonist from the antagonist, the amount of opioid antagonist being otherwise sufficient to counteract opioid agonist effect if administered parenterally.

The problem with all of the above schemes that incorporate opioid antagonists into the opioid preparation to dissuade abuse is that opioid antagonists themselves have side effects that may be disadvantageous. For example, nalorphine causes unpleasant reactions that range from anxiety, to “crazy feelings,” to hallucinations, respiratory depression and miosis. Seizures have been reported with naloxone, albeit infrequently, and in postoperative patients, pulmonary edema and ventricular fibrillation have been seen with high dosages. Naltrexone has been reported to have the capacity to cause hepatocellular injury when given in doses as low as fivefold or less of therapeutic doses. Nalmefene, although usually well tolerated, has been reported to cause nausea, vomiting and tachycardia in some individuals. Small doses of any of these opioid antagonists can also precipitate withdrawal in opioid addicted individuals even at low doses, a phenomenon that can be extremely dangerous depending upon where the addicted individual takes the drug.

There is a need, therefore, for novel methods of preventing opioid abuse which do not require the incorporation of opioid antagonists into the formulation.

### BRIEF SUMMARY OF THE INVENTION

The present invention provides an improved solid, oral dosage formulation that provides for the in vivo sustained-release of opioid compounds, and salts thereof, and in particular for the sustained-release of opioid analgesics, and which further inhibits the extraction of the opioid by common solvents from the formulation. The formulation dissuades abuse by limiting the ability of persons to extract the opioid from the formulation, such that the opioid cannot easily be concentrated for parenteral administration. Such an abuse-resistant formulation does not require incorporation of an opioid antagonist (albeit, an opioid antagonist may be added to the preparation to further dissuade abuse). The formulation comprises a simple mixture of a hydrophilic matrix-forming agent, ionic exchange resin, and one or more opioid compound(s). Such formulation may be prepared without the need for wet granulation of the mixture, drug loading of the resin, or the application of coating materials over the active component or the entire dosage form. Significantly improved formulations employ ionic exchange resins which are processed such that the particle size distribution of the resin is less than or equal to about 325 mesh, U.S. Standard Mesh Size, and the mean particle size of the resin particles is less than about 50 μm.

In particular, the present invention provides an improved formulation for the sustained release of oxycodone that hampers the extraction of the oxycodone from the formulation when extraction is by solvent extraction with commonly available household extraction solvents. In one embodiment of the present invention, the oxycodone formulation is an oxycodone sustained-release formulation which comprises a therapeutically effective amount of oxycodone, or salt thereof, in a matrix wherein the dissolution rate in vitro of the dosage form, when measured by the USP Basket Method at 100 rpm in 900 ml aqueous buffer (pH 1.2 for the first hour and 7.5 for hours 2 through 12) at 37° C. is between about 5 and 25% (by weight) oxycodone released over the first hour, between about 16 and 36% (by weight) oxycodone released after the second hour, between about 40 and 60% (by weight) oxycodone released after six hours, and between about 60 and 80% (by weight) oxycodone released after twelve hours. The release rate is independent of pH between about 1.2 and 7.5. Additionally the peak plasma level of oxycodone obtained in vivo occurs between five and six hours after administration of the dosage form.

Surprisingly, it has been found that formulations containing from about 5 to about 100 mg oxycodone may be manufactured to have such release rates when the formulation comprises between about 30 and 65% matrix-forming polymer, more preferably between 50-60% matrix-forming polymer, and between about 1 and 20% ion exchange resin. Significantly improved formulations containing 10 mg-30 mg of oxycodone hydrochloride contain between about 50 to about 60% matrix-forming polymer and between about 5 and about 15% ion exchange resin.

### DETAILED DESCRIPTION OF THE INVENTION

The present invention provides opioid formulations that are resistant to extraction of the opioid from the formulation, as compared to conventional opioid formulations, when extraction is performed using common household solvents such as isopropyl alcohol, vodka, white vinegar, hot water, peroxide, 0.01 N HCl and aqueous alcohol. The formulation is particularly useful when the structure of the opioid comprises a benzomorphan structure (lacking the C and E rings found in naturally occurring opioids), more particularly when the structure of the opioid comprises a morphinan structure (lacking the E ring found in naturally occurring opioids), and more particularly when the structure of the opioid comprises a morphine analog structure (having the A (aromatic), B (cyclohexane), C (cyclohexene), D (piperidine) and E (tetrahydrofuran) rings of morphine). Unexpectedly high resistance to extraction with such common household solvents is found when the formulation comprises oxycodone (having a methoxy group on the A ring of morphine at C3) as the opioid.

In a first aspect of the invention, there is disclosed a solid, oral dosage form comprising a therapeutically effective amount of opioid compound, or a salt thereof, between 30 and 65% of a matrix-forming polymer, more preferably between 50-60% matrix-forming polymer, and between 5 and 15% of a ionic exchange resin. Preferably the opioid compound included in the formulation is an opioid analgesic. As disclosed in U.S. patent application Ser. No. 09/626,584, the disclosure of which is incorporated in its entirety herein, it has been surprisingly found that a simple mixture of the matrix-forming agent with the opioid compound and ion-exchange resin, in the proportions disclosed, results in a formulation with improved opioid release kinetics without the need for, or recourse to, expensive coating procedures or wet granulation techniques. Such discovery is not taught by presently available opioid analgesic sustained-release preparations, and goes against conventional thought with respect to highly water soluble drugs (such as the opioid analgesics) which points toward the desirability of drug loading onto the resin, of coating drug-resin complexes, and which suggests that uncoated complexes provide only a relatively short delay of drug release (See, e.g., U.S. Pat. No. 4,996,047 to Kelleher et al.). Such formulation has now been found to provide surprising resistance to opioid extraction when extraction is attempted using commonly available household solvents such as isopropyl alcohol, vodka, white vinegar, hot water, peroxide, 0.01 N HCl and aqueous alcohol.

By the term “opioid,” it is meant a substance, whether agonist, antagonist, or mixed agonist-antagonist, which reacts with one or more receptor sites bound by endogenous opioid peptides such as the enkephalins, endorphins and the dynorphins. By the term “opioid analgesic” it is meant a diverse group of drugs, of natural, synthetic, or semi-synthetic origin, that displays opium or morphine-like properties. Opioid analgesics include, without limitation, morphine, heroin, hydromorphone, oxymorphone, buprenorphine, levorphanol, butorphanol, codeine, dihydrocodeine, hydrocodone, oxycodone, meperidine, methadone, nalbulphine, opium, pentazocine, propoxyphene, as well as less widely employed compounds such as alfentanil, allylprodine, alphaprodine, anileridine, benzylmorphine, bezitramide, clonitazene, cyclazocine, desomorphine, dextromoramide, dezocine, diampromide, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydroxypethidine, isomethadone, ketobemidone, levallorphan, levophenacylmorphan, lofentanil, meptazinol, metazocine, metopon, myrophine, narceine, nicomorphine, norpipanone, papvretum, phenadoxone, phenomorphan, phenazocine, phenoperidine, piminodine, propiram, sufentanil, tramadol, tilidine, and salts and mixtures thereof.

Matrix-forming polymers useful in the present invention may comprise any polymer not readily degradable by the body. Typical matrix-forming polymers useful in the present invention, include, without limitation, hydroxypropylmethyl cellulose (in particular having a molecular weight range of 50,000 to 1,250,000 daltons), ethylcellulose, methylcellulose, hydroxypropyl cellulose, hydroxyethyl cellulose, carboxymethyl cellulose calcium, sodium carboxymethylcellulose, hydroxypropylmethyl cellulose phthalate, cellulose acetate phthalate, carnauba wax and stearyl alcohol, carbomer, cetostearyl alcohol, cetyl alcohol, cetyl esters wax, guar gum, hydrogenated castor oil, magnesium aluminum silicate, maltodextrin, polyvinyl alcohol, polyvinyl chloride, polyethylene glycol, polyethylene glycol alginate, polymethacrylates, polyesters, polysaccharides, poloxamer, povidone, stearyl alcohol, glyceryl stearate, gelatin, acacia, dextran, alginic acid and sodium alginate, tragacanth, xanthan gum and zein. A preferred matrix-forming polymer is alkylcellulose-based, more particularly hydroxyalkylcellulose-based. Alkylcellulose matrix-forming polymers were found unexpectedly not only to improve the release profile of opioids when used in conjunction with numerous types of ionic exchange resins but also to provide a formulation with a significant resistance to extraction with isopropyl alcohol, vodka, white vinegar, hot water, peroxide, 0.01 N HCl and aqueous alcohol. The most efficacious matrix-forming polymers were found to be hydrophilic in nature.

Among the ionic exchange resins useful in the present invention, without limitation, are styrene-divinylbenzene copolymers (e.g. IRP-69, IR-120, IRA-400 and IRP-67—Rohm & Haas), copolymers of methacrylic acid and divinylbenzene (e.g. IRP-64 and IRP-88—Rohm & Haas), phenolic polyamines (e.g., IRP-58—Rohm & Haas), and styrene-divinylbenzene (e.g., colestyramine resin U.S.P.). The drug and resin should be oppositely charged such that the drug will bind to the resin when solubilized in the matrix formed by the matrix-former. As most opioid compounds are basic in nature, it is preferred that the ionic exchange resin be cationic in nature, and most preferably be strongly acidic in nature.

As discussed in U.S. patent application Ser. No. 09/626,584, it has been surprisingly found that micronization of the ionic resin particles, such that about 90% or more of the particles are less than about 325 mesh, U.S. Standard mesh size, or such that the particles have an mean particle size of less than about 50 μm, significantly improves the sustained release profile of a wide array of opioid compounds incorporated into a polymeric matrix, in particular a hydrophilic matrix. It is now found that such micronized ionic resin particles further provide increased resistance to extraction with commonly available household solvents such as isopropyl alcohol, vodka, white vinegar, hot water, peroxide, 0.01 N HCl and aqueous alcohol. A further aspect of the present invention therefore comprises a novel solid, oral, controlled release dosage form comprising a therapeutically effective amount of an opioid compound, or a salt thereof, between 30 and 65% of a matrix-forming polymer and between 5 and 15% ionic exchange resin having a mean particle size of less than about 50 μm and a particle size distribution such that not less than 90% of the particles pass through a 325 mesh sieve, US. Standard Sieve Size. In particular, the present inventor has found that strongly acidic cationic exchange resins, such as IRP-69 (Rohm & Hass), having a particle size of less than about 325 mesh (U.S. Standard mesh size) and/or a mean particle size of less than about 50 μm, more preferably less than about 44 μm, are particularly useful in formulating improved slow-release, extraction-resistant, oxycodone preparations, particularly when an alkylcellulose matrix-former is utilized.

The formulations of the present invention may include diluents, lubricants, glidants and additives, as known to those of ordinary skill in the art to improve compaction, augment swallowability, decrease gastrointestinal irritation, and generally to improve the pharmaceutical elegance of the final product. Among the diluents which may find application in the present formulations are, without limitation, lactose, microcrystalline cellulose, starch and pregelatinized starch, sucrose, compressible sugar and confectioner's sugar, polyethylene glycol, powdered cellulose, calcium carbonate, calcium sulfate, croscarmellose sodium, crospovidone, dextrates, dextrin, dextrose, fructose, glyceryl palmitostearate, kaolin, magnesium aluminum silicate, magnesium carbonate, magnesium oxide, maltodextrin, mannitol, dibasic calcium phosphate, tribasic calcium phosphate, sodium strach glycolate, sorbitol, and hydrogenated vegetable oil (type 1). Among the lubricants which may find application in the present formulations are, without limitation, stearic acid, calcium stearate, glyceryl monostearate, glyceryl palmitostearate, hydrogenated castor oil, hydrogenated vegetable oil (type 1), magnesium stearate, sodium stearyl fumarate, talc and zinc stearate. Suitable glidants, which may find application in the present formulations, are, without limitation, colloidal silicon dioxide, magnesium trisilicate, starch, talc, and tribasic calcium phosphate. Among the many additives that may find application in the present formulations are, without limitation, colorants, flavorants, sweetners, granulating agents, and coating agents such as cellulose acetate phthalate. A formulation of the present invention may comprise from 0.1-500 mg opioid compound, a matrix-forming polymer from 10-95% w/w, an ion exchange resin from 0.1-50% w/w, a diluent from 0-100% w/w, a glidant from 0-5% w/w and a lubricant from 0-20% w/w.

An advantage of the present formulations is that preparation of the formulations typically requires only industry standard equipment.

Another aspect of the present invention is a process for the preparation of a solid, controlled release, extraction-resistant oral dosage form comprising the step of incorporating an analgesically effective amount of an opioid analgesic, or salt thereof, in a bulk mixture comprising about 30 to about 65% of a matrix-forming polymer and about 5 to about 15% of a ionic exchange resin, thereby forming an admixture. Further disclosed is a process for the preparation of a solid, controlled release, extraction-resistant, oral dosage form comprising the step of incorporating an analgesically effective amount of oxycodone, or a salt thereof, in a bulk mixture comprising about 30 to about 65% of a matrix-forming polymer and about 5 to about 15% of an ionic exchange resin, wherein the dissolution rate in vitro, when measured by the USP Basket Method at 100 rpm in 900 ml aqueous buffer (pH 1.2 for the first hour and 7.5 for hours 2 through 12) at 37° C. is between about 5 and 25% (by weight) oxycodone released over the first hour, between about 16 and 36% (by weight) oxycodone released after the second hour, between about 40 and 60% (by weight) oxycodone released after six hours, and between about 60 and 80% (by weight) oxycodone released after twelve hours. The release rate is independent at pH between about 1.2 and 7.5. Additionally, the peak plasma level of oxycodone obtained in vivo occurs between five and six hours after administration of the dosage form.

### DETAILED DESCRIPTION OF THE PREFERRED EMBODIMENTS

Certain preferred embodiments of the present invention have been elucidated after numerous experiments.

The preferred matrix-forming polymer of the present formulations is an alkylcellulose, more preferably a C₁-C₆ hydroxyalkylcellulose. In a preferred dosage form the hydroxyalkylcellulose is selected from the group consisting of: hydroxypropylcellulose, hydroxypropylmethyl cellulose and hydroxyethylcellulose. While the ionic exchange resin of the present invention may be phenolic-based polyamine condensates or styrene-divinylbenzene co-polymers, it is preferred that the ionic exchange resin comprise a cationic exchange resin, in particular one which is sulfonated, to maximize charge-charge interactions between the resin and the opioids. Cationic exchange resins particularly useful in the present invention may comprise divinylbenzene co-polymers, such as a copolymer of divinylbenzene and styrene, or co-polymer of divinylbenzene and methacrylic acid, and the like. It is preferred that the ionic exchange resin comprise between 5 and 15% of the final dosage form, more preferably between about 7 and 10%. Preferably the final dosage form contains between about 40-65% matrix-forming polymer, more preferably between about 50-60%. The matrix-forming polymer, the opioid compound and ionic exchange resin are preferably admixed with one another in dry form, thus decreasing the time and expense involved in the formulation of a final dosage form. Preferably an oral dosage form is formed by, or in conjunction with, compression and shaping of the admixture. It is preferred, due to the advantageous drug release profile produced thereby, and the extraction-resistance of the preparation, that the ionic exchange resin have a mean particle size of less than about 50 μm and a particle size distribution such that not less than 90% of the particles pass through a 325 mesh sieve, U.S. Standard sieve size. Preferred opioid compounds useful in the present invention are selected from the group consisting of: butorphanol, codeine, dihydrocodeine, hydrocodone bitartrate, hydromorphone, meperidine, methadone, morphine, oxycodone hydrochloride, oxymorphone, pentazocine, propoxyphene hydrochloride and propoxyphene napsylate. Oxycodone preparations have been found to particularly resistant to extraction with isopropyl alcohol, vodka, white vinegar, hot water, peroxide, 0.01 N HCl and aqueous alcohol.

The present inventors have in particular discovered that fine particle size resin, having a particle size such that more than about 90% of the resin particles passes through a 325 mesh screen, U.S. Standard mesh size, significantly improves both the sustained release profile of the present formulations as compared to the regular particle size resins (e.g. Amberlite IRP-69M vs. Amberlite IRP-69) and its resistance to extraction by commonly available household solvents, in particular isopropyl alcohol, vodka, white vinegar, hot water, peroxide, 0.01 N HCl and aqueous alcohol. For example, biostudies of formulations using fine particle size resin suggest sustained-release formulations of the present invention may provide absorption equivalent to that obtained with oral oxycodone solutions with lower Cₘₐₓ.

Employment of the disclosed formulations with respect to the opioid oxycodone (dihydrohydroxycodeinone) hydrochloride has been found to be particularly advantageous. Oxycodone is a semisynthetic narcotic analgesic agent with actions, uses, and side effects similar to those of hydromorphone and morphine. Oxycodone is the opioid agent in at least 40 separate brand-name prescription medications. It is also found in a number of generic products. Oxycodone is prescribed for moderate to high pain relief associated with injuries, bursitis, dislocation, fractures, neuralgia, arthritis, and lower back and cancer pain. It is also used postoperatively and for pain relief after childbirth. Insurance companies typically cover the drug when used for the treatment of a covered illness. Typically formulated in conventional tablet form, this highly water soluble compound has a half-time of absorption of about 0.4 hours, a half-life of approximately 2 to 3 hours, and a duration of action of approximately 3 to 4 hours.

The United States Drug Enforcement Administration (DEA) has reported that oxycodone products have become drugs of abuse. The Office of National Drug Control Policy (ONDCP) reports that the number of oxycodone emergency cases increased nearly 36 percent in a single year, from 3,369 in January to June 1999 to 5,261 in January to June 2000. One oxycodone-containing product in particular, sold under the brand name OxyContin®, has been associated by the media with significant abuse potential. OxyContin® is an oral, controlled-release oxycodone that acts for 12 hours, making it one of the longest lasting oxycodone preparations on the market. OxyContin® (oxycodone hydrochloride controlled-release) tablets are supplied in 10 mg, 20 mg, 40 mg, and 80 mg tablet strengths for oral administration.

Oxycodone is frequently abused by addicts by administering the drug parenterally, most often by intravenous injection that is by accounts the most efficient means of maximizing a high. Since oxycodone is water soluble, crushed tablets can be dissolved in water, and an injectible solution is easily manufactured. Injection of solutions containing oxycodone allows the drug to be immediately available to receptor sites in the brain. Addicts indicate that intravenously administered oxycodone provides an immediate rush and wave of intense pleasure. Some addicts have described injected oxycodone as having similar effects to injected heroin. The pharmacological activities of oxycodone therefore have become attractive to some abuser populations as a substitute for heroin.

DEA agents report that heroin abusers are obtaining oxycodone tablets because the pharmaceutical preparations typically offer reliable strength and dosage levels. The DEA reports that some abusers have committed theft, armed robbery and fraud to obtain oxycodone tablets to be extracted for administration through a parenteral route. While no increase in illicit abuse of oxycodone was found in an April 2000 report of The Journal of the American Medical Association (JAMA) analyzing data stored in DEA's ARCOS (i.e., Automation of Reports and Consolidated Orders System) and DAWN MD (i.e., Drug Abuse Waning Network Medical Examiner) over the period 1990 to 1996, analysis of the same data since 1996 was seen to evidence significantly-increased abuse.

Oxycodone pharmaceuticals are Schedule II drugs under the Federal Comprehensive Drug Abuse Prevention and Control Act. Ironically, federal sentencing guidelines for diverted Schedule II pharmaceuticals are determined by the total weight of the tablets, not strength. Therefore, the penalty for distributing oxycodone illegally goes up as more excipient is added to the same concentration of oxycodone active.

A particularly useful formulation of oxycodone of the present invention, which has been found to effectively control pain in a wide variety of patients without significant pain breakthrough and which has been found by the present inventors to be resistant to extraction with commonly available household solvents such as isopropyl alcohol, vodka, white vinegar, hot water, peroxide, 0.01 N HCl and aqueous alcohol, comprises a solid, oral, controlled-release dosage form comprising a therapeutically effective amount of oxycodone, or a salt thereof, a matrix-forming polymer and an ionic exchange resin comprising a divinylbenzene copolymer, wherein the dissolution rate in vitro of the dosage form, when measured by the USP Basket Method at 100 rpm in 900 ml aqueous buffer (pH 1.2 for the first hour and 7.5 for hours 2 through 12) at 37° C. is between about 5 and 25% (by weight) oxycodone released over the first hour, between about 16 and 36% (by weight) oxycodone released after the second hour, between about 40 and 60% (by weight) oxycodone released after six hours, and between about 60 and 80% (by weight) oxycodone released after twelve hours. The in vitro release rate is independent of pH between about 1.2 and 7.5. Additionally, the peak plasma level of oxycodone obtained in vivo occurs between five and six hours after administration of the dosage form.

The following examples illustrate various aspects of the present invention. They are not, however, to be construed as limiting the claims in any manner whatsoever.

### EXAMPLE 1

Oxycodone hydrochloride 10 mg sustained-release dosage forms having the formulations given in Table I below were prepared as follows: oxycodone hydrochoride, USP, lactose NF (Flast Flo), and Amberlite IRP 69M fine particle size cationic exchange resin were run through a No. 20 mesh screen for delumping and were mixed for 10 minutes. Hydroxypropyl methylcellulose, USP, and Cab-O-Sil (M-5) (a glidant) was passed through a No. 20 mesh screen for delumping and then added to the drug powder blend. Mixing of the admixture was performed for 20 minutes. Stearic Acid NF (powder) (a lubricant) was passed through a No. 40 mesh screen and then added to the mixed batch. The batch was subsequently mixed for 3 minutes, the mixer sides wiped, and any adhering powder incorporated into the batch. The batch was then mixed for an additional 2 minutes and compressed to form tablets.

TABLE 1

| | FORMULA | FORMULA | FORMULA | FORMULA |
|---|---|---|---|---|
| INGREDIENT | 1 | 2 | 3 | 4 |
| Oxycodone | 10 mg/ | 10 mg/ | 10 mg/ | 10 mg/ |
| Hydrochloride | tablet | tablet | tablet | tablet |
| Lactose, NF | 27.8% w/w | 25.8% w/w | 31.1% w/w | 10.8% w/w |
| (Fast Flo) | | | | |
| Amberlite IRP | 5.0% w/w | 7.0% w/w | 6.7% w/w | 20.0% w/w |
| 69M Fine | | | | |
| Particle Size | | | | |
| Methocel | 55.0% w/w | 55.0% w/w | 50.0% w/w | 50.0% w/w |
| K100M | | | | |
| (Premium) CR | | | | |
| Cab-O-Sil | 0.5% w/w | 0.5% w/w | 0.5% w/w | 0.5% w/w |
| (M-5) | | | | |
| Stearic Acid, | 5.0% w/w | 5.0% w/w | 5.0% w/w | 5.0% w/w |
| NF (Powder) | | | | |
| Theoretical | 150 mg | 150 mg | 150 mg | 150 mg |
| Tablet Weight | | | | |

The in vitro release rates of formulations 1-4 were assessed by the USP Basket Method described herein above. Each of the formulations contained a total of 10 mg of oxycodone hydrochloride. The release rate of oxycodone from each of the preparations is set forth below in Table 2.

TABLE 2

| TIME | FORMULA | FORMULA | FORMULA 3 | FORMULA 4 |
|---|---|---|---|---|
| (HOURS) | 1 (% LA) | 2 (% LA) | (% LA) | (% LA) |
| 0 | 0 | 0 | 0 | 0 |
| 1 | 17.8 | 12.2 | 18.0 | 12.0 |
| 2 | 28.9 | 23.3 | 29.0 | 20.0 |
| 4 | 46.1 | 38.4 | 46.0 | 33.0 |
| 6 | 60.0 | 51.5 | 60.0 | 45.0 |
| 8 | 71.1 | 62.7 | 72.0 | 55.0 |
| 10 | 80.0 | 71.8 | 82.0 | 64.0 |
| 12 | 87.0 | 79.6 | 89.0 | 73.0 |

### EXAMPLE 2

Oxycodone hydrochloride 30 mg sustained-release dosage forms having the formulations given in Table 3 were prepared as follows: Lactose NF (Fast Flo) was passed through a No. 20 mesh screen for delumping and was mixed with the D and C Yellow No. 10 Aluminum Lake 6010 and FD and C Yellow No. 6 Aluminum Lake 5285 for 10 minutes. The lactose/color mix was then milled. Cab-O-Sil (M-5) (a glidant), oxycodone hydrochloride USP and Amberlite IRP-69M fine particle size were passed through a No. 20 mesh screen for delumping and were then mixed with the lactose/color blend for 10 minutes. Hydroxypropyl methylcellulose USP (Methocel K100M (premium) CR) was passed through a No. 20 mesh screen for delumping then added to the drug powder blend and mixed for 20 minutes. Stearic acid NF (powder) was passed through a No. 40 mesh screen and then added to the batch. The batch was mixed for 3 minutes, then the mixer sides and blades were wiped and adhering powder was incorporated into the batch. The batch was then mixed for an additional 2 minutes and compressed to form tablets.

TABLE 3

| INGREDIENT | FORMULA 5 | FORMULA 6 |
|---|---|---|
| Oxycodone Hydrochloride | 30 mg/tablet | 30 mg/tablet |
| Lactose, NF (Fast Flo) | 12.3% w/w | 14.5% w/w |
| Amberlite IRP 69M Fine Particle Size | 10.0% w/w | 5.0% w/w |
| Methocel K100M (Premium) CR | 55.0% w/w | 55.0% w/w |
| (hydroxylpropyl methylcellulose, | | |
| USP) | | |
| D and C Yellow No. 10 Aluminum | 0.4% w/w | 0.4% w/w |
| Lake 6010 | | |
| FD and C Yellow No. 6 Aluminum | 0.1% w/w | 0.1% w/w |
| Lake 5285 | | |
| Cab-O-Sil (M-5) | 0.5% w/w | 0.5% w/w |
| Stearic Acid, NF (Powder) | 5.0% w/w | 5.0% w/w |
| THEORETICAL TABLET WEIGHT | 150 mg | 150 mg |
| (approximate) | | |

The in vitro release rates of formulations 5 and 6, set forth in Table 3, were assessed by the USP Basket Method described herein above. Each of the formulations contained a total of 30 mg of oxycodone hydrochloride. The release rate of the oxycodone from each of the preparations is set forth below in Table 4.

TABLE 4

| TIME (HOURS) | FORMULA 1 (% LA) | FORMULA 2 (% LA) |
|---|---|---|
| 0 | 0 | 0 |
| 1 | 20 | 24.3 |
| 2 | 28 | 35.8 |
| 4 | 41 | 55.1 |
| 6 | 50 | 67.3 |
| 8 | 58 | 76.3 |
| 10 | 64 | 82.5 |
| 12 | 70 | N/A |

### EXAMPLE 3

The extractability of oxycodone from 40 mg oxycodone sustained-eased tablets having the following formulation:

| | |
|---|---|
| Oxycodone Hydrochloride | 40 mg |
| Lactose, NF (Fast Flo) | 16.1% w/w |
| Methocel K 100M | 45.% w/w |
| Amberlite IPR 69M | 12.5% w/w |
| Cab-O-Sil | 1.1% w/w |
| Stearic Acid, NF | 5.0% w/w |
| FD and C Yellow No 6 Aluminum Lake 5285 | 0.4% w/w |
| TOTAL TABLET WEIGHT | 200 mg |

was compared to the extractability of oxycodone from 40 mg OxyContin® sustained-release tablets. Commonly available household solvents were used, which solvents were isopropyl alcohol, vodka, white vinegar, hot water, hydrogen peroxide, 0.01 N HCl and aqueous alcohol (50:50 ethanol:water). Specifics of the solvents follow: isopropyl alcohol 70% concentration (Our Famil™ Isopropyl Rubbing Alcohol), vodka 100 proof (Smimoff® No. 57), white vinegar (Heinz® distilled), hot water (Barnseted Nanopure® water—used at ambient temperature and heated to 88° C.), hydrogen peroxide (Our Family™ 3% H₂O₂), 0.01 N HCl (prepared from a stock solution of 1 N HCl by dilution with water; stored at room temperature), aqueous alcohol (prepared by mixing 1 L ethanol with 1 L water; stored at room temperature).

Tablets were crushed and dissolved in 10 mL of household solvent by shaking at room temperature for 30 minutes. After centrifugation at 3000 rpm for twenty minutes, the supernatant was diluted in reconstitution solution either 5,000 or 50,000 fold. Reconstitution solution comprised 250 mL of mobile phase and 750 mL of 10 nM ammonium acetate. The mobile phase solvent mix was prepared by mixing 750 mL methanol, 1250 mL acetonitrile, and 300 mL of 10 mM ammonium acetate (sonified mixture). The diluted extracts were analyzed for oxycodone by LC/MS/MS (with a limit of quantitation of 2 ng/mL). Standard curves were generated covering a range of 2 to 500 ng/mL.

More specifically, one 40 mg tablet was added to a 16×100 mm silanized screw cap tube, and the tablet was crushed with a glass stirring rod until powder. Ten milliliters of extraction household solvent was added, the tubes capped and vortexed for two minutes. The resulting tubes were then shaken for thirty minutes on a rotary mixer and centrifuged for twenty minutes. The caps were subsequently removed, 0.02 ml of the extract supernatant removed, and the extract supernatant placed into 16×125 mm silanized tubes to which 9.98 mL of reconstitution solution was added. After vortexing, either a 5,000 or 50,000 fold dilution was made to 0.1 ml of diluted supernatant using reconstitution solution in a 13×100 mm silanized tube. Vortexing was once again undertaken. One tenth of a milliliter of sample from either the 5,000 or 50,000 fold dilution was transferred to a 13×100 mm silanized test tube, to which 0.1 mL internal standard and 0.8 mL reconstitution solution was added. After mixing, a sample of the mixture was transferred to autosampler vials and injected on LC/MS/MS. Chromatograms were integrated using MacQuan® software, and raw data was subsequently transferred into the Open VMS® on AlphaServer® Systems Oracle® database. Results were compared against a standard curve (linear range 2.0-500 ng/mL) to obtain a concentration (limit of quantitation approximately 2.0 ng/mL). A weighted ((1/x) where x=the concentration of the compound)) linear regression was used to determine slopes, intercepts and correlation coefficients.

Tables 5 (5,000 fold dilution) and 6 (50,000 fold dilution) set forth a comparison of the extraction of three tablets of such test formula of the present invention (T) against that of three tablets of OxyContin® (Oxy) for each solvent reference.

TABLE 5
(5,000 fold dilution)

| | Solvent: | | | | | | | | | | | | | |
|---|---|---|---|---|---|---|---|---|---|---|---|---|---|---|
| | | | | | | | 50% | | White | | | | Isopropyl | |
| | 0.1 N HCl | | Vodka | | Peroxide | | Ethanol | | Vinegar | | Hot Water | | Alcohol | |
| | Sample: | | | | | | | | | | | | | |
| | T | Oxy | T | Oxy | T | Oxy | T | Oxy | T | Oxy | T | Oxy | T | Oxy |
| Mean | 7.10 | 24.1 | 7.82 | 19.8 | 8.60 | 23.6 | 7.76 | 18.3 | 8.38 | 20.0 | 12.3 | 33.5 | 4.61 | 25.8 |
| Amount | | | | | | | | | | | | | | |
| Extracted: | | | | | | | | | | | | | | |
| In | | | | | | | | | | | | | | |
| mg/tablet | | | | | | | | | | | | | | |

| | | | | | | | |
|---|---|---|---|---|---|---|---|
| T as % | 29.5 | 39.5 | 36.4 | 42.4 | 41.9 | 36.7 | 17.9 |
| Oxy: | | | | | | | |

TABLE 6
(50,000 fold dilution)

| | Solvent: | | | | | | | | | | | | | |
|---|---|---|---|---|---|---|---|---|---|---|---|---|---|---|
| | | | | | | | 50% | | White | | | | Isopropyl | |
| | 0.1 N HCl | | Vodka | | Peroxide | | Ethanol | | Vinegar | | Hot Water | | Alcohol | |
| | Sample: | | | | | | | | | | | | | |
| | T | Oxy | T | Oxy | T | Oxy | T | Oxy | T | Oxy | T | Oxy | T | Oxy |
| Mean | 6.37 | 24.4 | 6.98 | 17.9 | 9.25 | 20.8 | 8.07 | 14.8 | 7.58 | 19.3 | 10.9 | 39.7 | 3.91 | 23.5 |
| Amount | | | | | | | | | | | | | | |
| Extracted: | | | | | | | | | | | | | | |
| in | | | | | | | | | | | | | | |
| mg/tablet | | | | | | | | | | | | | | |

| | | | | | | | |
|---|---|---|---|---|---|---|---|
| T as % | 26.1 | 39.0 | 44.5 | 54.5 | 39.3 | 27.5 | 16.6 |
| Oxy: | | | | | | | |

As indicated by the results in Tables 5 and 6, the test formulation (T) provided significantly more protection against oxycodone extraction from tablets made from the formulation than OxyContin® tablets when both were extracted with such common household solvents.

The following table shows the manufacturing processes for 10, 20 30 and 40 mg tablets according to the present invention

TABLE 7
Comparison of Formulation and Manufacturing Processes for 10, 20, 30 and 40 mg
Oxycodone Tablets
Brief Summary of Manufacturing Steps 10, 20, 30 and 40 mg Tablets.

| Step | | | |
|---|---|---|---|
| No. | 10 mg Tablets | 30 mg Tablets | 20 and 40 mg Tablets |
| 1) | — | Pass the Lactose NF (Fast Flo) | — |
| | | through a #20 mesh screen. | |
| 2) | — | Add Lactose NF (Fast Flo) and color | Add Lactose, NF (Fast Flo) and |
| | | (D and C Yellow No. 10 Aluminum | color to a bin and mix for 5 |
| | | Lake and FD and C Yellow No. 6 | minutes. |
| | | Aluminum Lake 5285) to the Sigma | |
| | | Mixer and mix for 10 minutes. | |
| 3) | — | Pass Step 2 through a mill | Pass Step 2 through a comil and |
| | | | add into a bin. |
| 4) | Pass the following through a #20 | Pass the following through a #20 | Pass the following through a |
| | mesh screen: | mesh screen: | comil and add to Step 3 bin: |
| | Oxycodone Hydrochloride, USP | Cab-O-Sil (M-5) | Cab-O-Sil (M-5) |
| | Lactose, NF (Fast Flo) | Oxycodone Hydrochloride, USP | Oxycodone Hydrochloride, USP |
| | Amberlite (IRP 69 M Fine Particle | Amberlite IRP 69M Fine Particle Size | Amberlite IRP 69M Fine Particle |
| | Size (Sodium Polystyrene | (Sodium Polystyrene Sulfonate, USP) | Size (Sodium Polystyrene |
| | Sulfonate, USP) | Step 3 | Sulfonate, USP) |
| | | | Methocel K100M (Premium CR) |
| | | | (Hydroxypropyl Methylcellulose, |
| | | | USP) |
| 5) | Mix Step 4 for 10 minutes in a | Mix Step 4 for 10 minutes in a Sigma | Mix Step 4 for 10 minutes. |
| | Sigma Mixer. | Mixer. | |
| 6) | Pass the following through a #20 | Pass the following through a #20 | Pass the following through a |
| | mesh screen: | mesh screen: | comil: |
| | Cab-o-sil (M-5) | Methocel K100M (Premium) CR | Stearic Acid, NF (Powder) |
| | Methocel K100M (Premium) CR | (Hydroxypropyl Methylcellulose, USP) | Proceed to Step 9. |
| | (Hydroxypropyl Methylcellulose, | | |
| | USP) | | |
| 7) | Add Step 6 to Step 5. Mix for 20 | Mix Step 6 for 20 minutes. | — |
| | minutes. | | |
| 8) | Pass the Stearic Acid, NF (Powder) | Pass the Stearic Acid, NF (Powder) | — |
| | through a #40 mesh screen. | through a #40 mesh screen. | |
| 9) | Add Step 8 to Step 7 in a Sigma | Add Step 8 to Step 7 in a Sigma | Mix Step 6 for 5 minutes. |
| | mixer and mix for 3 minutes. Wipe | mixer and mix for 3 minutes. Wipe | |
| | and incorporate any adhering | and incorporate any adhering | |
| | granulation from the sides and | granulation from the sides and blades | |
| | blades and mix an additional 2 | and mix an additional 2 minutes. | |
| | minutes. | | |
| 10) | Compress into tablets. | Compress into tablets. | Compress into tablets. |
| 11) | Package. | Package. | Package. |

The 10 mg manufacturing steps are shown independent of the 30 mg strenth due to lack of colorant in the 10 mg tablet.
— Manufacturing step is not included or is combined in another step.

While the invention has been described with respect to preferred embodiments, those skilled in the art will readily appreciate that various changes and/or modifications can be made to the invention without departing from the spirit or scope of the invention as defined by the appended claims.

1-6. (canceled)

7. In a method of treating a patient for pain or other condition where such patient is administered either oxycodone or oxycodone hydrochloride in a sustained release formulation and where it is possible for the patient to abuse the oxycodone or oxycodone hydrochloride by extraction of such medicament from the sustained release formulation through the use of solvents, the improvement which comprises administration of a solid, oral, controlled release dosage form consisting of a therapeutically effective amount of oxycodone or oxycodone hydrochloride or both, between about 30 and 65% by weight of a matrix-forming polymer selected from the group consisting of hydroxypropyl cellulose, hydroxypropylmethyl cellulose and hydroxyethyl cellulose and between about 1 and 20% by weight of a cationic exchange resin having a mean particle size of less than about 50 μm and a particle size distribution such that not less than 90% of the particles pass through a 325 mesh sieve, U.S. Standard Sieve Size, wherein the oxycodone or oxycodone hydrochloride or both, the polymer and the cationic exchange resin are admixed with one another in dry form and then compressed.

8. The method of claim 7 wherein the cationic exchange resin in the dosage form comprises a sulfonated polymer.

9. The method of claim 7 wherein the cationic exchange resin in the dosage form comprises a copolymer of divinyl-benzene and styrene.

10. The method of claim 7 wherein the cationic exchange resin in the dosage form comprises a copolymer of divinylbenzene and methacrylic acid.

11. The method of claim 7 wherein the cationic exchange resin in the dosage form comprises phenolic-based polyamine condensates.
